(19) United States
(12) Patent Application Publication
(10) Pub. No.: US 2005/0272687 A1
(43) Pub. Date: Dec. 8, 2005

(54) Stable S-adenosyl-l-methionine

(76) Inventor: Rolland F. Hebert, Seattle, WA (US)

Correspondence Address: ROLLAND HEBERT, 427 BELLEVUE AVE E. SUITE 301, SEATTLE, WA, 98102, US

(21) Appl. No.: 11/136,271

(22) Filed: May 24, 2005

Related U.S. Application Data
(60) Provisional application No. 60/578,030, filed on Jun. 8, 2004.

(51) Int. Cl. A61K 31/7076; A61K 31/716; A61K 31/405; A61K 31/19

(52) U.S. Cl. 514/46; 514/55; 514/57; 514/419; 514/574

(57) ABSTRACT

Stable conjugates of S-adenosyl-1-methionine, methods for their synthesis and methods for their uses are described. The conjugates according to the invention are very stable and are valuable for use as active constituents in pharmaceutical compositions.

### CROSS-REFERENCES TO RELATED APPLICATIONS

This application claims the benefit of Provisional Patent Application Ser. No. 60/578,030 filed on June, 8, 2004. This patent application is partially related to provisional patent 60/472,149 filed on May 21, 2003 which is incorporated herein in its entirety but is abandoned.

### FIELD OF THE INVENTION

The present invention relates to new conjugates of S-adenosyl-1-methionine with tryptophan, fumaric acid, chitosan, dextran, azelaic acid and carboxy methyl cellulose, new conjugates of existing salts of S-adenosyl-1-methionine and synthetic methods.

### TECHNICAL FIELD

This patent relates to new salts of S-adenosyl-1-methionine (known as SAM-e) with tryptophan, fumaric acid, chitosan, azelaic acid, dextran and carboxy methyl cellulose and new salts of salts of S-adenosyl-1-methionine with carboxy methyl cellulose, fumaric acid, and azelaic, the processes for obtaining them and to therapeutic uses of these new salts, more particularly, the invention relates to compositions deriving from the reaction between S-adenosyl-1-methionine, and tryptophan, azelaic acid, fumaric acid, chitosan, dextran and carboxy methyl cellulose, their production process and pharmaceutical compositions that contain them as active principles.

### BACKGROUND OF THE INVENTION

S-adenosyl-1-methionine is a naturally occurring substance that is present in all living organisms and has a number of very important biological functions. S-adenosyl-1-methionine exists in two important diastereomeric forms as (S,S) S-adenosyl-1-methionine and (R,S) S-adenosyl-1-methionine. Among these functions are the following: methyl group donor in transmethylation reactions (it is the sole methyl group donor in such reactions-including methylation of DNA and RNA, proteins, hormones, catechol and indoleamines and phosphatidylethanolamine to phosphatidylcholine); it is a substrate of an enzyme lyase that converts S-adenosyl-1-methionine to the molecule methylthioadenosine and homoserine; it is an aminobutyric chain donor to tRNA; it is an aminoacidic chain donor in the biosynthesis of biotin; S-adenosyl-1-methionine, after decarboxylation, is the donor of aminopropyl groups for the biosynthesis of neuroregulatory polyamines spermidine and spermine. (Zappia et al (1979) Biomedical and Pharmacological roles of Adenosylmethionine and the Central Nervous System, page 1, Pergamon Press. NY.)

S-adenosyl-1-methionine is a naturally occurring substance that is present in all living organisms and has a number of very important biological functions. S-adenosyl-1-methionine exists in two important diastereomeric forms as (S,S) S-adenosyl-1-methionine and (R,S) S-adenosyl-1-methionine. Among these functions are the following: methyl group donor in transmethylation reactions (it is the sole methyl group donor in such reactions-including methylation of DNA, proteins, hormones, catechol and indoleamines and phosphatidylethanolamine to phosphatidylcholine); it is a substrate of an enzyme lyase that converts S-adenosyl-1-methionine to the molecule methylthioadenosine and homoserine; it is an aminobutyric chain donor to tRNA; it is an aminoacidic chain donor in the biosynthesis of biotin; S-adenosyl-1-methionine, after decarboxylation, is the donor of aminopropyl groups for the biosynthesis of neuroregulatory polyamines spermidine and spermine. (Zappia et al (1979) Biomedical and Pharmacological roles of Adenosylmethionine and the Central Nervous System, page 1, Pergamon Press. NY.)

The (R,S) S-adenosyl-1-methionine diastereomer has been reported to have an opposite activity to that of the (S,S)) S-adenosyl-1-methionine diastereomer. (Borchardt and Wu, Journal of Medicinal Chemistry, 1976, Vol. 19, No. 9, pp 1099 to 1103. Thus, it may be of particular interest to develop compositions that have one or the other of the diastereomers in higher concentrations relative to the other when clinical conditions so dictate. Thus, since the (R,S) S-adenosyl-1-methionine is a potent methyltransferase inhibitor, it would be desirable to have it in highest concentrations when one wishes to inhibit such methyltransferases in conditions which may require such inhibition, for example, cancer.

S-adenosyl-1-methionine has been used clinically for more than twenty years in the treatment of liver disease (Friedel H, Goa, K. L., and Benfield P., (1989) S-Adenosyl-1-methionine: a review of its pharmacological properties and therapeutic potential in liver dysfunction and affective disorders in relation to its physiological role in cell metabolism. Drugs. 38, 389-416), arthritis (Di Padova C, (1987) S-adenosyl-1-methionine in the treatment of osteoarthritis: review of the clinical studies. Am J. Med. 83, (Suppl. 5), 6-65), and depression (Kagan, B, Sultzer D. L., Rosenlicht N and Gemer R. (1990) Oral S-adenosylmethionine in depression: a randomized, double-blind, placebo-controlled trial. Am. J. Psychiatry 147, 591-595.) Alzheimer's patients have reduced cerebral spinal fluid levels of S-adenosyl-1-methionine (Bottiglieri et al, (1990) Cerebrospinal fluid S-adenosyl-1-methionine in depression and dementia: effects of treatment with parenteral and oral S-adenosyl-1-methionine. J. Neurol. Neurosurg. Psychiatry 53, 1096-1098.) In a preliminary study, S-adenosyl-1-methionine was able to produce cognitive improvement in patients with Alzheimer's disease. (Bottiglieri et al (1994) The clinical potential of admetionine (S-adenosyl-1-methionine) in neurological disorders. Drugs 48, 137-152.) S-adenosyl-1-methionine brain levels in patients with Alzheimer's disease are also severely decreased. (Morrison et al, (1996)

Brain S-adenosylmethionine levels are severely decreased in Alzheimer's disease, Journal of Neurochemistry, 67, 1328-1331. Patients with Parkinson's disease have also been shown to have significantly decreased blood levels of S-adenosyl-1-methionine. (Cheng et al, (1997) Levels of L-methionine S-adenosyltransferase activity in erythrocytes and concentrations of S-adenosylmethionine and S-adenosylhomocysteine in whole blood of patients with Parkinson's disease. Experimental Neurology 145, 580-585.) Oral S-adenosyl-1-methionine administration to patients with and without liver disease has resulted in increases in liver glutathione levels. (Vendemiale G et al, Effect of oral S-adenosyl-1-methionine on hepatic glutathione in patients with liver disease. Scand J Gastroenterol 1989; 24: 407-15. Oral administration of S-adenosyl-1-methionine to patients suffering from intrahepatic cholestasis had improvements in both the pruritus as well as the biochemical markers of cholestasis. (Giudici et al, The use of admetionine (S-adenosyl-1-methionine) in the treatment of cholestatic liver disorders. Meta-analysis of clinical trials. In: Mato et al editors. Methionine Metabolism: Molecular Mechanism and Clinical Implications. Madrid: CSIC Press; 1992 pp 67-79.) Oral S-adenosyl-1-methionine administration to patients suffering from primary fibromyalgia resulted in significant improvement after a short term trial. (Tavoni et al, Evaluation of S-adenosylmethionine in Primary Fibromyalgia The American Journal of Medicine, Vol 83 (suppl 5A), pp 107-110, 1987.) Lee Hong Kyu disclosed in a patent application WO02092105 (November, 21, 2002) that S-adenosyl-1-methionine could be used to treat diabetes and insulin resistance. A recently published evidence report entitled “S-adenosyl-1-methionine for the treatment of depression, osteoarthritis and liver disease” provides both safety and clinical efficacy data for this important biomolecule. (Evidence Report number 64, US Department of Health and Human Services, Public Health Service, Agency for Healthcare Research and Quality. October 2002.

S-adenosyl-1-methionine is clinically useful in many apparently unrelated areas because of its important function in basic metabolic processes. One of its most striking clinical uses is in the treatment of alcoholic liver cirrhosis that, until now, remained medically untreatable. Mato et al, in 1999, demonstrated the ability of oral S-adenosyl-1-methionine in alcoholic liver cirrhosis to decrease the overall mortality and/or progression to liver transplant by 29% vs 12% as compared with a placebo treated group. (Mato et al, (1999) S-adenosylmethionine in alcohol liver cirrhosis: a randomized, placebo-controlled, double blind, multi-center clinical trial. Journal of Hepatology, 30, 1081-1089.) The extensive clinical use of S-adenosyl-1-methionine has proven its efficacy as well as its absence of toxicity in a number of different clinical conditions. Indeed, further basic science as well as clinical studies on this very important molecule may elucidate new uses for S-adenosyl-1-methionine in medicine.

S-adenosyl-1-methionine has been used clinically in the treatment of liver disease (Friedel H, Goa, K. L., and Benfield P., (1989), S-adenosyl-1-methionine: a review of its pharmacological properties and therapeutic potential in liver dysfunction and affective disorders in relation to its physiological role in cell metabolism. Drugs. 38, 389-416), arthitis (Di Padova C, (1987), S-adenosyl-1-methionine in the treatment of osteoarthritis: review of the clinical studies. Am J. Med. 83, (Suppl. 5), 6-65), and depression (Kagan, B, Sultzer D. L., Rosenlicht N and Gerner R. (1990), Oral S-adenosyl-1-methionine in depression: a randomized, double blind, placebo-controlled trial. Am. J. Psychiatry 147, 591-595.) Alzheimer's patients have reduced cerebral spinal fluid levels of S-adenosyl-1-methionine (Bottiglieri et al, (1990), Cerebrospinal fluid S-adenosyl-1-methionine in depression and dementia: effects of treatment with parenteral and oral S-adenosyl-1-methionine. J. Neurol. Neurosurg. Psychiatry 53, 1096-1098.) In a preliminary study, S-adenosyl-1-methionine was able to produce cognitive improvement in patients with Alzheimer's disease. (Bottiglieri et al (1994), The clinical potential of admetionine (S-adenosyl-1-methioinine) in neurological disorders. Drugs 48, 137-152.)

S-adenosyl-1-methionine brain levels in patients with Alzheimer's disease are also severely decreased. (Morrison et al, (1996), Brain S-adenosyl-1-methionine levels are severely decreased in Alzheimer's disease, Journal of Neurochemistry, 67, 1328-1331.) Patients with Parkinson's disease have also been shown to have significantly decreased blood levels of S-adenosyl-1-methionine. (Cheng et al, (1997), Levels of L-methionine S-adenosyltransferase activity in erythrocytes and concentrations of S-adenosyl-1-methionine and S-adenosylhomocysteine in whole blood of patients with Parkinson's disease. Experimental Neurology 145, 580-585.)

S-adenosyl-1-methionine levels in patients treated with the antineoplastic drug methotrexate are reduced. Neurotoxicity associated with this drug may be attenuated by co-administration of S-adenosyl-1-methionine. (Bottiglieri et al (1994), The Clinical Potential of Ademetionine (S-adenosyl-1-methionine) in neurological disorders, Drugs, 48 (2), 137-152.)

Cerebral spinal fluid levels of S-adenosyl-1-methionine have been investigated in HIV AIDS dementia Complex/HIV encephalopathy and found to be significantly lower than in non-HIV infected patients. (Keating et al (1991), Evidence of brain methyltransferase inhibition and early brain involvement in HIV positive patients Lancet: 337:935-9.)

De La Cruz et al have shown that S-adenosyl-1-methionine, chronically administered, can modify the oxidative status in the brain by enhancing anti-oxidative defenses. (De La Cruz et al, (2000), Effects of chronic administration of S-adenosyl-1-methionine on brain oxidative stress in rats. Naunyn-Schmiedeberg's Archives Pharmacol 361: 47-52.) This is similar to results obtained with S-adenosyl-1-methionine in liver and kidney tissue. Thus S-adenosyl-1-methionine would be useful as an antioxidant

Oral S-adenosyl-1-methionine administration to patients with and without liver disease has resulted in increases in liver glutathione levels. (Vendemiale G et al, (1989), Effect of oral S-adenosyl-1-methionine on hepatic glutathione in patients with liver disease. Scand J Gastroenterol; 24: 407-15. Oral administration of S-adenosyl-1-methionine to patients suffering from intrahepatic cholestasis had improvements in both the pruritus as well as the biochemical markers of cholestasis. (Giudici et al, The use of admethionine (S-adenosyl-1-methionine) in the treatment of cholestatic liver disorders. Meta-analysis of clinical trials. In: Mato et al editors. Methionine Metabolism: Molecular Mechanism and Clinical Implications. Madrid: CSIC Press; 1992 pp 67-79.)

Oral S-adenosyl-1-methionine administration to patients suffering from primary fibromyalgia resulted in significant improvement after a short-term trial. (Tavoni et al, Evaluation of S-adenosylmethioine in Primary Fibromaylgia. The American Journal of Medicine, Vol 83 (suppl 5A), pp 107-110, 1987.) S-adenosyl-1-methionine has been used for the treatment of osteoarthritis as well. (Koenig B. A long-term (two years) clinical trial with S-adenosyl-1-methionine for the treatment of osteoarthritis. The American Journal of Medicine, Vol 83 (suppl 5A), Nov. 20, 1987 pp 89-94)

S-adenosyl-1-methionine also attenuates the damage caused by tumor necrosis factor alpha and can also decrease the amount of tumor necrosis factor alpha secreted by cells. Consequently, conditions in which this particular inflammatory factor is elevated would benefit from the administration of S-adenosyl-1-methionine. (Watson W H, Zhao Y, Chawla R K, (1999) Biochem J Aug 15; 342 (Pt 1):21-5. S-adenosyl-1-methionine attenuates the lipopolysaccharide-induced expression of the gene for tumour necrosis factor alpha.) S-adenosyl-1-methionine has also been studied for its ability to reduce the toxicity associated with administration of cyclosporine A, a powerful immunosuppressor. (Galan A, et al, Cyclosporine A toxicity and effect of the S-adenosyl-1-methionine, Ars Pharmaceutica, 40:3; 151-163, 1999.)

S-adenosyl-1-methionine, incubated in vitro with human erythrocytes, penetrates the cell membrane and increases ATP within the cell thus restoring the cell shape. (Friedel et al, S-adenosyl-1-methionine: A review of its pharmacological properties and therapeutic potential in liver dysfunction and affective disorders in relation to its physiological role in cell metabolism, Drugs 38 (3):389-416, 1989).

S-adenosyl-1-methionine has been studied in patients suffering from migraines and found to be of benefit. (Friedel et al, S-adenosyl-1-methionine: A review of its pharmacological properties and therapeutic potential in liver dysfunction and affective disorders in relation to its physiological role in cell metabolism, Drugs 38 (3): 389-416, 1989)

Belli et al in an article entitled “S-adenosylmethionine prevents total parenteral nutrition-induced cholestasis in the rat”, Journal of Hepatology 1994; 21: 18-23 showed that S-adenosyl-1-methionine was able to prevent cholestasis resulting from total parenteral nutrition by maintaining liver plasma membrane enzymatic activity via preservation of the membrane lipid environment.

S-adenosyl-1-methionine has been administered to patients with peripheral occlusive arterial disease and was shown to reduce blood viscosity, chiefly via its effect on erythrocyte deformability.

Garcia P et al in “S-adenosylmethionine: A drug for the brain?”, IV th Workshop on Methionine Metabolism: Molecular Mechanisms and Clinical Implications”, Symposium held on March 1-5, Granada, Spain, 1998, reported that S-adenosyl-1-methionine was able to increase the number of muscarinic receptors in the brains of rats treated chronically with S-adenosyl-1-methionine. Muscarinic receptors in the brain, especially in the hippocampus, are important in learning and memory. In a standard eight arm radical maze test, treated animals were able to out-perform age matched older untreated animals. Young aged matched S-adenosyl-1-methionine treated animals were also able to out-perform young non-treated animals showing S-adenosyl-1-methionine's ability to increase memory even in young animals. The conclusions drawn were that S-adenosyl-1-methionine is able to improve memory not only in adult aged animals but also in young animals thus making S-adenosyl-1-methionine an eligible candidate therapy for the treatment of memory impairment and learning difficulties.

S-adenosyl-1-methionine, however, presents certain difficult problems in terms of its stability at ambient temperature that result in degradation of the molecule to undesirable degradation products as well as to the epimerization of the (S,S) S-adenosyl-1-methionine to (R,S) S-adenosyl-1-methionine. S-adenosyl-1-methionine has therefore been the subject of numerous patents directed both towards the obtaining of new stable salts, and towards the provision of preparation processes which can be implemented on an industrial scale. All citations referenced in this patent application are incorporated herein in their entirety.

Many organic compounds exist in optically active forms, i.e., they have the ability to rotate the plane of plane-polarized light. In describing an optically active compound, the prefixes D and L or R and S are used to denote the absolute configuration of the molecule about its chiral center. The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these compounds, called stereoisomers, are identical except that they are mirror images of one another. A specific stereoisomer may also be referred to as an enantiomer, and a mixture of such isomers is often called an enantiomeric mixture. A 50:50 mixture of enantiomers is referred to as a racemic mixture. A compound with more than one chiral center is a diastereomer. S-adenosyl-1-methionine is a diastereomer.

Stereochemical purity is of importance in the field of pharmaceuticals, where 12 of the 20 most prescribed drugs exhibit chirality. A case in point is provided by the L-form of the beta-adrenergic blocking agent, propranolol, which is known to be 100 times more potent than the D-enantiomer.

Furthermore, optical purity is important since certain isomers may actually be deleterious rather than simply inert. For example, it has been suggested that the D-enantiomer of thalidomide was a safe and effective sedative when prescribed for the control of morning sickness during pregnancy, and that the corresponding L-enantiomer was a potent teratogen. S-adenosyl-1-methionine is a naturally occurring substance that is present in all living organisms and has a number of very important biological functions. Among these functions are the following: methyl group donor in transmethylation reactions (it is the sole methyl group donor in such reactions-including methylation of DNA, proteins, hormones, catechol and indoleamines and phosphatidylethanolamine to phosphatidylcholine); it is a substrate of an enzyme lyase that converts S-adenosyl-1-methionine to the molecule methylthioadenosine and homoserine; it is an aminobutyric chain donor to tRNA; it is an aminoacidic chain donor in the biosynthesis of biotin; S-adenosyl-1-methionine, after decarboxylation, is the donor of amino propyl groups for the biosynthesis of neuroregulatory polyamines spermidine and spermine. (Zappia et al (1979), Biomedical and Pharmacologcial roles of Adenosylmethionine and the Central Nervous System, page 1, Pergamon Press. NY.)

S-adenosyl-1-methionine is commercially available using fermentation technologies that result in S-adenosyl-1-methionine formulations varying between 60 and 80% purity. (That is, the final product contains 60-80% of the active or (S,S)-S-adenosyl-1-methionine and 20-40% of the inactive (in terms of transmethylation) or (R,S)-S-adenosyl-1-methionine.) (Gross, A., Geresh, S., and Whitesides, G m (1983) Appl. Biochem. Biotech. 8, 415.) Enzymatic synthetic methodologies have been reported to yield the inactive isomer (in terms of transmethylation) in concentrations exceeding 60%. (Matos, J R, Rauschel F M, Wong, C H. S-adenosyl-1-methionine: Studies on Chemical and Enzymatic Synthesis. Biotechnology and Applied Biochemistry 9, 39-52 (1987). S-adenosyl-1-methionine may also be produced using bacteria to produce the desired molecule.

Yeast and bacteria as a rule produce solely the (S,S) S-adenosyl-1-methionine diastereomer. However, during the extraction as well as the purification process, epimerization of the (S,S) S-adenosyl-1-methionine diastereomer to the (RS) S-adenosyl-1-methionine form takes place. An example of bacterial fermentation is disclosed in U.S. patent application 20040175805, Leonhartsberger et al, Sep. 9, 2004. However, Leonhartsberger do not disclose or recognize the importance of stabilization of the S-adenosyl-1-methionine molecule at lower temperature to prevent or slow down the rate of epimerization.

Enantiomeric separation technologies have been reported to resolve the pure active diastereomer of S-adenosyl-1-methionine. (Matos, J R, Rauschel F M, Wong, C H. S-adenosyl-1-methionine: Studies on Chemical and Enzymatic Synthesis. Biotechnology and Applied Biochemistry 9, 39-52 (1987; Hoffman, Chromatographic Analysis of the Chiral and Covalent Instability of S-adenosyl-1-methionine, Biochemistry 1986, 25 4444-4449: Segal D and Eichler D, The Specificity of Interaction between S-adenosyl-1-methionine and a nucleolar 2-O-methyltransferase, Archives of Biochemistry and Biophysics, Vol. 275, No. 2, December, pp. 334-343, 1989) Newer separation technologies exist to resolve enantiomers and diastereomers on a large commercial production scale at a very economic cost. In addition, it would be conceivable to synthesize the biologically active diastereomer using special sterioselective methodologies but this has not been accomplished to date.

De la Haba first showed that the sulfur is chiral and that only one of the two possible configurations was synthesized and used biologically. (De la Haba et al J. Am. Chem. Soc. 81, 3975-3980, 1959) Methylation of RNA and DNA is essential for normal cellular growth. This methylation is carried out using S-adenosyl-1-methionine as the sole or major methyl donor with the reaction being carried out by a methyltransferase enzyme. Segal and Eichler showed that the enzyme bound (S,S)-S-adenosyl-1-methionine 10 fold more tightly than the biologically inactive (R,S)-S-adenosyl-1-methionine thus demonstrating a novel binding stereospecificity at the sulfur chiral center. Other methyltransferases have been reported to bind (R,S)-S-adenosyl-1-methionine to the same extent as (S,S)-S-adenosyl-1-methionine and thus (R,S)-S-adenosyl-1-methionine could act as a competitive inhibitor of that enzyme. (Segal D and Eichler D, The Specificity of Interaction between S-adenosyl-1-methionine and a nucleolar 2-O-methyltransferase, Archives of Biochemistry and Biophysics, Vol. 275, No. 2, December, pp. 334-343, 1989; Borchardt R T and Wu Y S, Potential inhibitors of S-adenosyl-1-methionine-dependent methyltransferases. Role of the Asymmetric Sulfonium Pole in the Enzymatic binding of S-adenosyl-1-methionine, Journal of Medicinal Chemistry, 1976, Vol 19, No. 9, 1099-1103.)

Borchardt and Wu, in an article entitled “Potential Inhibitors of S-adenosyl-1-methionine-dependent methyltransferases. 5. Role of the Asymmetric Sulfonium Pole in the Enzymatic Binding of Adenosyl-L-methionine”, Journal of Medicinal Chemistry, 1976, Vol. 19, No. 9, pp 1099-1103, report that the (+)-SAM (no longer used nomenclature for (R,S)-S-adenosyl-1-methionine) is a potent inhibitor of enzyme-catalyzed transmethylation reactions. Since transulferation and methylation reactions are the hallmark of S-adenosyl-1-methionine's mechanism of action, it would be prudent to use S-adenosyl-1-methionine with as enriched a concentration of (S,S)-S-adenosyl-1-methionine in any pharmaceutical composition as possible since the (R,S)-S-adenosyl-1-methionine diastereomer may be inhibitory to the desired action of (S,S)-S-adenosyl-1-methionine.

Detich et al in an article entitled “The methyl donor S-adenosyl-1-methionine inhibits active demethylation of DNA; a candidate novel mechanism for the pharmacological effects of S-adenosylmethionine.” J Biol. Chem. 2003 Jun. 6; 278(23):20812-20, point out the tumor protective mechanism of S-adenosyl-1-methionine and the importance of intracellular S-adenosyl-1-methionine concentrations in cancer prevention. Presumably this is due to the ability of S-adenosyl-1-methionine to prevent or reverse global genomic DNA hypomethylation. Indeed, DNA hypomethylation is a hallmark of cancer cells and the correction of this hypomethylation leads to proper gene expression and reversal or prevention of cancer.

However, in light of the known inability of (R,S)-S-adenosyl-1-methionine to participate in methylation or transulfuration reactions (indeed, it inhibits these reactions), it becomes increasingly apparent that S-adenosyl-1-methionine compositions should contain the least amount of (R,S)-S-adenosyl-1-methionine possible when the activity one wishes to use clinically relates to methylation.

S-adenosyl-1-methionine (whether in its optically pure diastereomeric form or in defined non-racemic ratios of (S,S)-S-adenosyl-1-methionine to (R,S)-S-adenosyl-1-methionine or as a racemic mixture) presents certain difficult problems in terms of its stability at ambient temperature that result in degradation of the molecule to undesirable degradation products as well as to epimerization to its less desirable form (R,S)-S-adenosyl-1-methionine. S-adenosyl-1-methionine (and thus its diastereomers) must be further stabilized since they exhibit intramolecular instability that causes the destabilization and breakdown of the molecule at both high as well as ambient temperatures.

Najm et al in BMC Musculoskelet Disord. 2004 Feb. 26; 5(1):6 confronted the problem of S-adenosyl-1-methionine epimeric instability during a double-blind cross-over trial for the treatment of osteoartritis using S-adenosyl-1-methionine. During the course of the clinical trial, the authors noted that the S-adenosyl-1-methionine they used at the beginning of the trial had epimerized to a ratio of (S,S) S-adenosyl-1-methionine/(R,S) S-adenosyl-1-methionine of 45%/55% respectively. Thus, the trial had to be halted until new batches of S-adenosyl-1-methionine could be made to continue the trial. This is a problem for all salts of S-adenosyl-1-methionine and clearly poses a quality control issue for drug development. The present patent solves, to some extent, the quality control issues inherent in this unstable molecule. Thus, by controlling the temperature of the extraction and purification steps during the manufacturing process, the rate of epimerization of S-adenosyl-1-methionine from (S,S) S-adenosyl-1-methionine to (R,S) S-adenosyl-1-methionine can be slowed down. Interestingly and unexpectedly, the inventor has discovered that if the concentration of (S,S) S-adenosyl-1-methionine to (R,S) S-adenosyl-1-methionine is very high during the successful manufacturing process, the rate of epimerization once the molecule has been dried (either by freeze drying or other methodologies known in the art) will be slowed as well. Thus, for example, if the salified S-adenosyl-1-methionine is dried by spray drying, a process accomplished at high temperatures, epimerization of the S-adenosyl-1-methionine molecule will occur advantageously. Spray drying results in a (S,S) S-adenosyl-1-methionine to (R,S) S-adenosyl-1-methionine ratio of 70% to 30% respectively. Once on the shelf, such a product will further epimerize to 50%/50% (S,S) S-adenosyl-1-methionine to (R,S) S-adenosyl-1-methionine or even lower as shown in the Najm paper.

All attempts at synthetic methodologies to manufacture S-adenosyl-1-methionine have resulted in racemic mixtures of 50%/50% (S,S) S-adenosyl-1-methionine to (R,S) S-adenosyl-1-methionine. Stereochemical synthesis of S-adenosyl-1-methionine has not yet been accomplished. However, the very same problem of epimerization would be raised during the manufacturing process. To overcome the epimerization of the molecule from (S,S) S-adenosyl-1-methionine to (R,S) S-adenosyl-1-methionine one would be required to control the temperature of the synthetic reaction as well as purification (in event that the synthesis does not result in 100% (S,S) S-adenosyl-1-methionine) and the salification process. Thus the inventor anticipates that the problems of stereochemical synthesis of S-adenosyl-1-methionine will be overcome. The inventor contemplates halting of the epimerization of the S-adenosyl-1-methionine using temperature to control the rate of epimerization in the same manner as disclosed herein with regards to yeast or bacterial fermentation to obtain the S-adenosyl-1-methionine.

S-adenosyl-1-methionine has therefore been the subject of many patents directed both towards obtaining new stable salts, and towards the provision of preparation processes that can be implemented on an industrial scale. The present patent thus envisions the use of any of the salts of S-adenosyl-1-methionine already disclosed in the prior art in order to stabilize the diastereomeric forms of S-adenosyl-1-methionine. Examples of such salts disclosed in the prior art include, but not limited to, the following: a lipophilic salt of S-adenosyl-1-methionine of the formula S-adenosyl-1-methionine.sup.n+[R—CO—NH—(CH.sub.2).sub.2-SO.sup.-.sub.3]. sub.n in which R—CO is a member selected from the group consisting of C.sub.12-C.sub.26 saturated and unsaturated, linear and branched acyl and C.sub.12-C.sub.26 cycloalkyl-substituted acyl, and n is an integer from 3 to 6 according to the S-adenosyl-1-methionine charge; double salts corresponding to the formula S-adenosyl-1-methionine.sup.+.HSO.sub.4.sup.-.H.sub.2 SO.sub.4.2 CH.sub.3 C.sub.6H.sub.4 SO.sub.3H.; salts (S, S)-s-adenosyl-1-methionine with sulphonic acids selected from the group consisting of methanesulphonic, ethanesulphonic, 1-n-dodecanesulphonic, 1-n-octadecanesulphonic, 2-chloroethanesulphonic, 2-bromoethanesulphonic, 2-hydroxyethanesulphonic, 3-hydroxypropanesulphonic, d-, 1-,d, 1-10-camphorsulphonic, d-, 1-,d, 1-3-bromocamphor-10-sulphonic, cysteic, benzenesulphonic,p-chlorobenzenesulphonic, 2-mesitylbenzenesulphonic, 4-biphenylsulphonic, 1-naphthalenesulphonic, 2-naphthalenesulphonic, 5-sulphosalicylic, p-acetylbenzenesulphonic, 1,2-ethanedisulphonic, methanesulphonic acid, ethanesulphonic acid, 1-n-dodecanesulphonic acid, 1-n-octadecanesulphonic acid, 2-chloroethanesulphonic acid, 2-bromoethanesulphonic acid,2-hydroxyethanesulphonic acid, d-,1-,d,1-10-camphorsulphonic acid, d-,1-,d,1-3-bromocamphor-10-sulphonic acid, cysteic acid, benzenesulphonic acid, 3-hydroxypropanesulphonic acid, 2-mesitylbenzenesulphonic acid, p-chlorobenzenesulphonic acid,4-biphenylsulphonic acid, 2-naphthalenesulphonic acid, 5-sulphosalicylic acid, 1,2-ethanedisulphonic acid, p-acetylbenzenesulphonic acid, 1-naphthalenesulphonic acid, o-benzenedisulphonic and chondroitinesulphuric acids, and double salts of said acids with sulphuric acid; S-adenosyl-1-methionine or a pharmaceutically acceptable salt thereof and an effective amount of a lithium salt selected from the group consisting of lithium chloride, lithium bromide, lithium iodide, lithium sulfate, lithium nitrate, lithium phosphate, lithium borate, lithium carbonate, lithium formate, lithium acetate, lithium citrate, lithium succinate and lithium benzoate; water-soluble salt of a bivalent or trivalent metal is a member selected from the group consisting of calcium chloride, ferric chloride, magnesium chloride, and magnesium sulfate; the salt of S-adenosyl-1-methionine is a member selected from the group consisting of salts of S-adenosyl-1-methionine with hydrochloric acid, sulfuric acid, phosphoric acid, formic acid, acetic acid, citric acid, tartaric acid, and maleic acid; and a double salt of S-adenosyl-1-methionine with said acids; a salt of S-adenosyl-1-methionine and a water-soluble polyanionic substance selected from the group consisting of a polyphosphate, metaphosphate, polystyrene sulfonate, polyvinyl sulfonate, polyvinyl sulfate, polyvinyl phosphate, and polyacrylate wherein the stoichiometric ratio of mols of S-adenosyl-1-methionine to gram-equivalent of the polyanionic substance is from 0.1:1 to 0.5; a salt of S-adenosyl-1-methionine wherein the polyanionic substance is a polyphosphate, para-polystyrene sulfonate or metaphosphate; a salt of the general formula: S-adenosyl-1-methionine.nR(O).sub.m (SO.sub.3H)p (I) where m can be zero or 1; n is 1.5 when p is 2, and is 3 when p is 1; R is chosen from the group consisting of alkyl, phenylalkyl and carboxyalkyl, in which the linear or branched alkyl chain contains from 8 to 18 carbon atoms, and in particular for producing S-adenosyl-1-methionine salts of sulphonic acids, or of sulphuric acid esters, or of dioctylsulphosuccinic acid. However the more preferred salts of the S-adenosyl-1-methionine diastereomers are chosen from the group consisting of salts (either single or double) of S-adenosyl-1-methionine diastereomers with sulfuric acid, p-toluenesulfonic acid, and 1,4-butanedisulfonate.

### PRIOR ART

There exist numerous patents disclosing many new salts of S-adenosyl-1-methionine but none discloses the use of tryptophan, fumaric acid, chitosan, dextran or carboxy methyl cellulose to stabilize S-adenosyl-1-methionine its diastereomers or non-racemic mixtures thereof. U.S. Pat. No. 3,893,999, Fiecchi, Jul. 8, 1975, discloses a new salt of S-adenosyl-1-methionine made with tri-p-toluensulphonate but not the use of tryptophan, fumaric acid, chitosan, dextran or carboxy methyl cellulose to stabilize S-adenosyl-1-methionine its diastereomers or non-racemic mixtures thereof. U.S. Pat. No. 3,954,726, Fiecchi, May 4, 1976, discloses double salts of S-adenosyl-1-methionine but does not disclose the use of tryptophan, fumaric acid, chitosan, dextran or carboxy methyl cellulose to stabilize S-adenosyl-1-methionine its diastereomers or non-racemic mixtures thereof. U.S. Pat. No. 4,028,183, Fiecchi, Jun. 7, 1977, discloses, among others, p-toluene sulfonate as a means to stabilize the S-adenosyl-1-methionine molecule but does not disclose the use of tryptophan, fumaric acid, chitosan, dextran or carboxy methyl cellulose to stabilize S-adenosyl-1-methionine, its diastereomers or non-racemic mixtures thereof. United States Patent. 4,057,686, Fiecchi, Nov. 8, 1977, discloses stable salts of S-adenosyl-1-methionine but does not disclose the use of chitosan, dextran or carboxy methyl cellulose to stabilize S-adenosyl-1-methionine, its diastereomers or non-racemic mixtures thereof.

In U.S. Pat. No. 4,465,672, Gennari, Aug. 14, 1984, discloses new S-adenosyl-1-methionine salts but does not disclose the use of chitosan, dextran or carboxy methyl cellulose to stabilize S-adenosyl-1-methionine, its diastereomers or defined non-racemic mixtures thereof. Gennari in U.S. Pat. No. 4,465,672 also does not disclose purification or extraction of the S-adenosyl-1-methionine at lower temperatures as indicated in this present patent to prevent or slow down the epimerization process once the S-adenosyl-1-methionine has been released from the yeast cells.

U.S. Pat. No. 4,543,408, Gennari, Sep. 24, 1985, discloses new S-adenosyl-1-methionine salts but does not disclose the use of chitosan, dextran or carboxy methyl cellulose to stabilize S-adenosyl-1-methionine, its diastereomers or non-racemic mixtures thereof. U.S. Pat. No. 4,558,122, Gennari, Dec. 10, 1985, discloses new S-adenosyl-1-methionine salts but does not disclose the use of chitosan, dextran or carboxy methyl cellulose to stabilize S-adenosyl-1-methionine, its diastereomers or non-racemic mixtures thereof. U.S. Pat. No. 4,990,606, Gennari, Feb. 5, 1991, discloses new salts of S-adenosyl-1-methionine but does not disclose the use of chitosan, dextran or carboxy methyl cellulose to stabilize S-adenosyl-1-methionine, its diastereomers or non-racemic mixtures thereof. U.S. Pat. No. 5,102,791, Gennari, Apr. 7, 1992, discloses, among others, a 1,4 butanedisulfonate salt of S-adenosyl-1-methionine but not the use of chitosan, dextran or carboxy methyl cellulose to stabilize S-adenosyl-1-methionine, its diastereomers or non-racemic mixtures thereof. Gennari also does not disclose the process of lowering the temperature of the extraction or purification steps in order to prevent further epimerization of the (S,S) S-adenosyl-1-methionine to (RS) S-adenosyl-1-methionine, nor does Gennari disclose the non-racemic ratios of the (S,S) S-adenosyl-1-methionine to (RS) S-adenosyl-1-methionine as disclosed in the present patent application.

In U.S. Pat. No. 5,114,931, Gennari, May 19, 1992, discloses injectable S-adenosyl-1-methionine salts but does not disclose the use of chitosan to stabilize S-adenosyl-1-methionine, its diastereomers or non-racemic mixtures thereof. U.S. Pat. No. 5,128,249, Gennari, Jul. 7, 1992, discloses new S-adenosyl-1-methionine salts but does not disclose the use of chitosan, dextran or carboxy methyl cellulose to stabilize S-adenosyl-1-methionine, its diastereomers or non-racemic mixtures thereof. U.S. Pat. No. 3,707,536, Haid et al, Dec. 26, 1972, discloses a new S-adenosyl-1-methionine bisulfate salt but not the use of chitosan, dextran or carboxy methyl cellulose to stabilize S-adenosyl-1-methionine, its diastereomers or non-racemic mixtures thereof. U.S. Pat. No. 4,109,079 Kawahara, et al., Aug. 22, 1978, discloses new stable S-adenosyl-1-methionine salts but does not disclose the use of tryptophan, fumaric acid, chitosan, dextran or carboxy methyl cellulose to stabilize S-adenosyl-1-methionine, its diastereomers or non-racemic mixtures thereof.

In U.S. Pat. No. 4,242,505, Kawahara, et al. Dec. 30, 1980, disclose new stabilizing salts of S-adenosyl-1-methionine but does not disclose the use of tryptophan, fumaric acid, chitosan, dextran or carboxy methyl cellulose to stabilize S-adenosyl-1-methionine, its diastereomers or non-racemic mixtures thereof. U.S. Pat. No. 4,369,177, Kozaki et al, Jan. 18, 1983, disclose stable compositions of S-adenosyl-1-methionine and S-adenosyl-1-methionine salts using a salt of a bivalent or trivalent metal but does not disclose the use of tryptophan, fumaric acid, chitosan, dextran or carboxy methyl cellulose salts of S-adenosyl-1-methionine, its diastereomers or non-racemic mixtures thereof or of other S-adenosyl-1-methionine salts. U.S. Pat. No. 5,166,328 Kurobe, et al. Nov. 24, 1992 entitled “S-adenosylmethionine derivatives” does not disclose the use of tryptophan, fumaric acid, chitosan, dextran or carboxy methyl cellulose to stabilize S-adenosyl-1-methionine, its diastereomers or non-racemic mixtures thereof.

U.S. Pat. No. 2,969,353, Shunk et al, Jan. 24, 1962, discloses a method for the preparation of S-adenosyl-1-methionine and a stable salt of S-adenosyl-1-methionine but not the use of tryptophan, fumaric acid, chitosan, dextran or carboxy methyl cellulose to stabilize S-adenosyl-1-methionine, its diastereomers or non-racemic mixtures thereof. U.S. Pat. No. 4,764,603, Zappia, et al. Aug. 16, 1988, discloses the use of new salts of S-adenosyl-1-methionine but does not disclose the use of tryptophan, fumaric acid, chitosan, dextran or carboxy methyl cellulose to stabilize S-adenosyl-1-methionine, its diastereomers or non-racemic mixtures thereof. U.S. Pat. No. 5,073,546, Zappia, et al. Dec. 17, 1991, discloses new salts of S-adenosyl-1-methionine but does not disclose the use of tryptophan, fumaric acid, chitosan, dextran or carboxy methyl cellulose to stabilize S-adenosyl-1-methionine, its diastereomers or non-racemic mixtures thereof. U.S. Pat. No. 6,117,849, Zimmermann, et al. Sep. 12, 2000, discloses the use of S-adenosyl-1-methionine complexed with nucleosides as HIV inhibitors but does not disclose the use of tryptophan, fumaric acid, chitosan, dextran or carboxy methyl cellulose to stabilize S-adenosyl-1-methionine, its diastereomers or non-racemic mixtures thereof. In addition, none of the above mentioned patents disclose the use of carboxy methyl cellulose to further stabilize salts of S-adenosyl-1-methionine, its diastereomers or non-racemic mixtures thereof that are cited in the above mentioned patents. No prior art (with the exception of the Bema patent application) discusses methodologies to prevent or slow down the inevitable epimerization of the molecule. However, Berna also does not accomplish the stabilization of the S-adenosyl-1-methionine molecule either. While Berna discloses that the ratio of (S,S) S-adenosyl-1-methionine to (R,S) S-adenosyl-1-methionine remains stable over a four month period using their techniques, it is now known that the ratio of (S,S) S-adenosyl-1-methionine to (R,S) S-adenosyl-1-methionine resulting from the Berna technique also declines over time.

Thus it is one object of the present invention to provide new salts of S-adenosyl-1-methionine, its diastereomers or non-racemic mixtures thereof using chitosan, it is another object of the present invention to provide new salts of S-adenosyl-1-methionine, its diastereomers or non-racemic mixtures thereof, using dextran. It is yet another object of the present invention to provide new salts of S-adenosyl-1-methionine, its diastereomers or non-racemic mixtures thereof using carboxy methyl cellulose. It is another object of the present invention to provide new salts of S-adenosyl-1-methionine, its diastereomers or non-racemic mixtures thereof using tryptophan or fumaric acid. It is a further object of the present invention to provide new conjugates of existing salts of s-adenosyl-1-methionine, their diastereomers or non-racemic mixtures thereof using tryptophan or fumaric acid.

It is a further object of the present invention to provide new conjugates of any existing salts of s-adenosyl-1-methionine, their diastereomers or non-racemic mixtures thereof using carboxy methyl cellulose.

It is another object of the present invention to provide a method of extracting and purification of the S-adenosyl-1-methionine at temperatures between 1-7 degrees C. to prevent, halt or slow down the epimerization of (S,S) S-adenosyl-1-methionine to (RS) S-adenosyl-1-methionine.

Administration of new compositions of S-adenosyl-1-methionine with dextran (a macromolecule composed of glucose subunits and have been used in clinical medicine for a long time.) and chitosan, and semi synthetic polymer, carboxy methyl cellulose of the present invention would have significant utility over a wide range of disorders or conditions associated with low levels of S-adenosyl-1-methionine. The new compositions of S-adenosyl-1-methionine with tryptophan, an amino acid, fumaric acid (unsaturated dicarboxylic acid), azelaic acid, chitosan, dextran, and/or carboxy methyl cellulose, would be more stable at room temperature over a longer period of time than current salts of S-adenosyl-1-methionine.

These new compositions of S-adenosyl-1-methionine with tryptophan, fumaric acid, azelaic acid, chitosan, dextran or carboxy methyl cellulose would not cause gastrointestinal upset often associated with the current S-adenosyl-1-methionine salts. In this regard, and in view of the molecular instability of S-adenosyl-1-methionine at room temperature over time, it has been suggested that a more ideal composition of S-adenosyl-1-methionine would be able to withstand the conditions of room temperature over long periods of time which would duplicate the shelf life conditions under which these new S-adenosyl-1-methionine compositions would be stored.

Additionally, new salts or conjugates of salts of S-adenosyl-1-methionine, their diastereomers and defined non-racemic mixtures thereof would also have significant utility over a wide range of disorders or conditions associated with low levels of S-adenosyl-1-methionine.

Tryptophan, fumaric acid, azelaic acid. chitosan, dextran, carboxy methyl cellulose and S-adenosyl-1-methionine and their salts are all available commercially and are considered non-toxic.

Accordingly, there is need in the art for new, stable compositions of S-adenosyl-1-methionine as well as methods related to the use of such compositions to increase blood and other tissue and fluid levels of S-adenosyl-1-methionine and to treat conditions which result from low blood and tissue levels of S-adenosyl-1-methionine. There is also a need in the art for synthetic routes to make such new compositions. The author of this present invention fulfills these needs and provides further related advantages.

### SUMMARY OF THE INVENTION

Briefly stated, the present invention discloses new, stable compositions of S-adenosyl-1-methionine, its diastereomers or defined non-racemic mixtures thereof, with tryptophan, fumaric acid, azelaic acid, chitosan, dextran or carboxy methyl cellulose. This present invention also discloses new, stable compositions of salts of S-adenosyl-1-methionine, their diastereomers or defined non-racemic mixtures thereof conjugated with carboxy methyl cellulose, methods for the use thereof and synthetic methods for their preparation.

Additionally, the present invention discloses new more stable S-adenosyl-1-methionine 1,4 butanedisulfonate salts, its diastereomers or defined non-racemic mixtures and their 1,4 butanedisulfonate salts when the S-adenosyl-1-methionine is extracted and purified under specific temperature conditions.

These new compositions of S-adenosyl-1-methionine with tryptophan, fumaric acid, azelaic acid. chitosan, dextran or carboxy methyl cellulose and/or a S-adenosyl-1-methionine salt with carboxy methyl cellulose or azelaic acid of this present invention have utility in increasing global genomic DNA, intracellular, blood and other tissue or fluid levels of S-adenosyl-1-methionine, as well as treating or preventing a wide variety of conditions associated with low blood or other tissue or fluid levels of S-adenosyl-1-methionine.

In another embodiment, a method is disclosed to prevent, halt or slow down the epimerization of (S,S) S-adenosyl-1-methionine to (R,S) S-adenosyl-1-methionine during the extraction and purification process by keeping the temperature at which these procedures are carried out between 1 and 7 degrees C. This is an important step since it is another embodiment of the current patent to provide compositions of stable salts and conjugates of stable salts of optically pure and defined non-racemic ratios of (S,S) S-adenosyl-1-methionine to (R,S) S-adenosyl-1-methionine.

In another embodiment, a new composition of S-adenosyl-1-methionine with tryptophan, fumaric acid, chitosan, dextran or carboxy methyl cellulose or a S-adenosyl-1-methionine salt with carboxy methyl cellulose is administered to a warm blooded animal in need thereof to prevent or treat a condition associated with low levels of S-adenosyl-1-methionine.

Thus in one embodiment, a new composition of S-adenosyl-1-methionine with tryptophan, fumaric acid, azelaic acid, chitosan, dextran or carboxy methyl cellulose or a S-adenosyl-1-methionine salt with carboxy methyl cellulose or azelaic acid is administered to a warm-blooded animal in need thereof to increase S-adenosyl-1-methionine levels. In another embodiment, a new composition of S-adenosyl-1-methionine with tryptophan, fumaric acid, chitosan, dextran or carboxy methyl cellulose or a S-adenosyl-1-methionine salt with carboxy methyl cellulose is administered to a warm blooded animal in need thereof to prevent or treat a condition associated with low levels of S-adenosyl-1-methionine.

In yet a further embodiment, a new composition of S-adenosyl-1-methionine with tryptophan, fumaric acid, azelaic acid, chitosan, dextran or carboxy methyl cellulose or a S-adenosyl-1-methionine salt with carboxy methyl cellulose or azelaic acid is administered to a warm blooded animal in need thereof to prevent and or treat the following conditions: aging, aging of the skin, Alzheimer's disease, arthritis, both as an anti-inflammatory as well as to promote new cartilage formation and prevent cartilage destruction, nerve damage associated with HIV/AIDS, anxiety, obsessive compulsive disorder, attention deficit disorder and ADHD, sleep regulation, organ preservation for transplant industry, treatment of dyslipidemias, excess sebum production, migraines, prevention and treatment of bile dysfunction caused by pregnancy and use of contraceptive medications, cancer, depression, acute and chronic liver disease, cirrhosis of the liver, ischemic reperfusion injury of stroke as well as organ ischemic reperfusion in transplant technology, Parkinson's disease, memory disturbances, intrahepatic cholestasis, inflammation, diabetes, pain and to counteract the decrease in S-adenosyl-1-methionine caused by various cancer and immunosuppressive drugs.

In yet a further embodiment, various synthetic methodologies for making the compositions of the present invention are disclosed.

The author of the present invention has surprisingly discovered that new, more stable compositions of S-adenosyl-1-methionine can be made with tryptophan, fumaric acid, azelaic acid, chitosan, dextran or carboxy methyl cellulose. The author of the present invention has surprisingly discovered that new, more stable compositions of any existing salt S-adenosyl-1-methionine can be made with carboxy methyl cellulose or azelaic acid. These new salts with tryptophan, fumaric acid, azelaic acid, chitosan, dextran or carboxy methyl cellulose or new conjugates of any existing S-adenosyl-1-methionine salt with carboxy methyl cellulose or azelaic acid provide steric hindrance to the unstable S-adenosyl-1-methionine molecule resulting in a much more stable molecule over time.

### DETAILED DESCRIPTION OF THE INVENTION

As mentioned above, this invention is generally directed to new compositions of S-adenosyl-1-methionine, its diastereomers or non-racemic mixtures thereof, with tryptophan, fumaric acid, chitosan, dextran or carboxy methyl cellulose or any S-adenosyl-1-methionine salt, its diastereomers or non-racemic mixtures thereof, with carboxy methyl cellulose. Such new compositions of S-adenosyl-1-methionine with chitosan, dextran or carboxy methyl cellulose or any S-adenosyl-1-methionine salt conjugated with carboxy methyl cellulose when administered to a warm blooded animal in need thereof have utility in the prevention or treatment of conditions associated with low levels of S-adenosyl-1-methionine in warm blooded animals, including humans.

The author of the present invention has surprisingly discovered that new, more stable compositions of S-adenosyl-1-methionine can be made with tryptophan, fumaric acid, azelaic acid, chitosan, dextran or carboxy methyl cellulose or a S-adenosyl-1-methionine salt with carboxy methyl cellulose. These new salts with tryptophan, fumaric acid, chitosan, dextran or carboxy methyl cellulose or a S-adenosyl-1-methionine salt with carboxy methyl cellulose provide steric hindrance to the unstable S-adenosyl-1-methionine molecule resulting in a much more stable molecule over time. It is to be noted that any salt that stabilizes S-adenosyl-1-methionine and already mentioned in the scientific or patent literature can be used in the present invention and then further stabilized since, to date, no perfect stable S-adenosyl-1-methionine has ever been synthesized.

S-adenosyl-1-methionine is commercially available using fermentation technologies that result in S-adenosyl-1-methionine formulations varying between 60 and 80% purity. (That is, the final product contains 60-80% of the active or (S, S)-S-adenosyl-1-methionine and 20-40% of the inactive or (R, S)-S-adenosyl-1-methionine.) (Gross, A., Geresh, S., and Whitesides, Gm (1983) Appl. Biochem. Biotech. 8, 415.) Enzymatic synthetic methodologies have been reported to yield the inactive isomer in concentrations exceeding 60%. (Matos, J R, Rauschel F M, Wong, C H. S-Adenosylmethionine: Studies on Chemical and Enzymatic Synthesis. Biotechnology and Applied Biochemistry 9, 39-52 (1987). A recent U.S. patent application 20020188116 Deshpande, Pandurang Balwant; et al. Dec. 12, 2002 entitled “Chemical synthesis of S-adenosyl-L-methionine with enrichment of (S,S)-isomer.” discloses methodology to synthesize S-adenosyl-1-methionine but does not disclose any methodology to stabilize the molecule once its synthesized. In addition, Deshpande et al do not disclose the process of controlling the temperature of the synthetic reaction. U.S. Patent Application 20020173012 Berna, Marco; et al. Nov. 21, 2002 entitled “Process for the preparation of pharmaceutically acceptable salts of (R,S)-S-adenosyl-L-methionine” disclose a process for the preparation of a relatively purified biologically active diastereomer (S,S) S-adenosyl-1-methionine (97%) but does not disclose stabilization of the S-adenosyl-1-methionine molecule using tryptophan, fumaric acid, chitosan, dextran or carboxy methyl cellulose or an S-adenosyl-1-methionine salt with carboxy methyl cellulose. The Berna patent application 20020173012 also does not disclose making 1,4 butanedisulfonate salts of the S-adenosyl-1-methionine diastereomers. S-adenosyl-1-methionine1,4 butanedisulfonate salts would have significant advantage over the S-adenosyl-1-methionine tosylate or other salts since S-adenosyl-1-methionine1,4 butanedisulfonate salts are better absorbed orally than any other salts. (Knoll internal documents provided to Food and Drug Administration of US government, Mar. 2, 1998). In addition, S-adenosyl-1-methionine1,4 butanedisulfonate salt has been more extensively studied in humans than other forms of S-adenosyl-1-methionine.

S-adenosyl-1-methionine (whether in its optically pure diastereomeric form or in an enantiomeric or racemic mixture) presents certain difficult problems in terms of its stability at ambient temperature that result in degradation of the molecule to undesirable degradation products. S-adenosyl-1-methionine (and thus its diastereomer s) must be further stabilized since it exhibits intramolecular instability that causes the destabilization and breakdown of the molecule at both high as well as ambient temperatures. S-adenosyl-1-methionine has therefore been the subject of many patents directed both towards obtaining new stable salts, and towards the provision of preparation processes that can be implemented on an industrial scale.

As used herein, the term “conditions” includes diseases, injuries, disorders, indications and/or afflictions that are associated with decreased levels of S-adenosyl-1-methionine and methylation of DNA. The term “treat” or “treatment” means that the symptoms associated with one or more conditions associated with low levels of S-adenosyl-1-methionine are alleviated or reduced in severity or frequency and the term “prevent” means that subsequent occurrences of such symptoms are avoided or that the frequency between such occurrences is prolonged.

The term “defined non-racemic” mixture or ratio of (S,S) S-adenosyl-1-methionine to (R,S) S-adenosyl-1-methionine includes compositions of the present invention wherein the defined non-racemic ratio of (S,S) S-adenosyl-1-methionine to (R,S) S-adenosyl-1-methionine is about 1%: 100% to about 100%: 1% by weight

The term “defined non-racemic” mixture or ratio of (S,S) S-adenosyl-1-methionine to (R,S) S-adenosyl-1-methionine salts excludes compositions of the Berna patent application 20020173012 but includes the defined non-racemic ratio of (S,S) S-adenosyl-1-methionine to (R,S) S-adenosyl-1-methionine is about 1%: 96.99% to about 96.99%: 1% by weigh when single or double salts of the diastereomers are made using sulfuric and paratoluensulfphonic acids.

The term “defined non-racemic” mixture or ratio of (S,S) S-adenosyl-1-methionine to (R,S) S-adenosyl-1-methionine includes also any existing salts of S-adenosyl-1-methionine wherein the defined non-racemic ratio of (S,S) S-adenosyl-1-methionine to (R,S) S-adenosyl-1-methionine is about 80%: 100% to about 100%: 80% by weight and when the S-adenosyl-1-methionine is extracted and purified at temperatures between 2 and 7 degrees Centigrade.

Typical oral dosages for the treatment of the conditions listed above lie in the range of from 100 mg to 1600 mg or greater per day given in divided doses orally or by other routes of delivery currently used. Typical IM or IV dosages are in the range of between 200 mg and 1200 mg daily continuous or divided.

The amount of tryptophan, fumaric acid, azelaic acid, chitosan, dextran or carboxy methyl cellulose that may be used to stabilize the S-adenosyl-1-methionine, its diastereomers or non-racemic mixtures thereof, in the synthetic process of this patent can range from 1% of the weight of the S-adenosyl-1-methionine (or its salts) (its diastereomers or define non-racemic mixtures or ratios) to be stabilized to 100% of the weight of the S-adenosyl-1-methionine to be stabilized. For reasons of economy, the least amount of tryptophan, fumaric acid, chitosan, dextran or carboxy methyl cellulose needed to stabilize the S-adenosyl-1-methionine, its diastereomers or non-racemic mixtures thereof is the preferred quantity. The solutions that result during the synthetic process can be dried by any other methods than freeze drying that are all well known in the art. After drying, a stable S-adenosyl-1-methionine powder results.

Owing to their simple conception and low costs, the procedures described in this invention easily lend themselves to working out methods of preparation on an industrial scale.

Thus it is one object of the present invention to provide new salts of S-adenosyl-1-methionine, its diastereomers or non-racemic mixtures thereof using chitosan, it is another object of the present invention to provide new salts of S-adenosyl-1-methionine, its diastereomers or non-racemic mixtures thereof, using dextran and it is yet another object of the present invention to provide new salts of S-adenosyl-1-methionine, its diastereomers or non-racemic mixtures thereof using carboxy methyl cellulose or azelaic acid. It is another object of the present invention to provide new salts of S-adenosyl-1-methionine, its diastereomers or non-racemic mixtures thereof using tryptophan, azelaic acid, or fumaric acid.

Administration of new compositions of S-adenosyl-1-methionine with dextran and chitosan, and carboxy methyl cellulose of the present invention would have significant utility over a wide range of disorders or conditions associated with low levels of S-adenosyl-1-methionine. The new compositions of S-adenosyl-1-methionine with tryptophan, fumaric acid, chitosan, dextran, and/or carboxy methyl cellulose, would be more stable at room temperature over a longer period of time than current salts of S-adenosyl-1-methionine. These new compositions of S-adenosyl-1-methionine with tryptophan, fumaric acid, chitosan, dextran or carboxy methyl cellulose would not cause gastrointestinal upset often associated with the current S-adenosyl-1-methionine salts. In this regard, and in view of the molecular instability of S-adenosyl-1-methionine at room temperature over time, it has been suggested that a more ideal composition of S-adenosyl-1-methionine would be able to withstand the conditions of room temperature over long periods of time which would duplicate the shelf life conditions under which these new S-adenosyl-1-methionine compositions would be stored.

Tryptophan, fumaric acid, chitosan, dextran, carboxy methyl cellulose and S-adenosyl-1-methionine and their salts are all available commercially and are considered non-toxic.

S-adenosyl-1-methionine is commercially available using fermentation technologies that result in S-adenosyl-1-methionine formulations varying between 60 and 80% purity. (That is, the final product contains 60-80% of the active or (S, S)-S-adenosyl-1-methionine and 2040% of the inactive or (R, S)-S-adenosyl-1-methionine.) (Gross, A., Geresh, S., and Whitesides, Gm (1983) Appl. Biochem. Biotech. 8, 415.) Enzymatic synthetic methodologies have been reported to yield the inactive isomer in concentrations exceeding 60%. (Matos, J R, Rauschel F M, Wong, C H. S-Adenosylmethionine: Studies on Chemical and Enzymatic Synthesis. Biotechnology and Applied Biochemistry 9, 39-52 (1987). A recent U.S. patent application 20020188116 Deshpande, Pandurang Balwant; et al. Dec. 12, 2002 entitled “Chemical synthesis of S-adenosyl-L-methionine with enrichment of (S,S)-isomer.” discloses methodology to synthesize S-adenosyl-1-methionine but does not disclose any methodology to stabilize the molecule once its synthesized. U.S. Patent Application 20020173012 Berna, Marco; et al. Nov. 21, 2002 entitled “Process for the preparation of pharmaceutically acceptable salts of (R,S)-S-adenosyl-L-methionine” disclose a process for the preparation of a relatively purified biologically active diastereomer (S,S) S-adenosyl-1-methionine (97%) but does not disclose stabilization of the S-adenosyl-1-methionine molecule using tryptophan, fumaric acid, chitosan, dextran or carboxy methyl cellulose. In addition, Berna et al have not disclosed the use of 1,4 butanedisulfonate to make the salt of S-adenosyl-1-methionine.

S-adenosyl-1-methionine (whether in its optically pure diastereomeric form or in an enantiomeric or racemic mixture) presents certain difficult problems in terms of its stability at ambient temperature that result in degradation of the molecule to undesirable degradation products. S-adenosyl-1-methionine (and thus its diastereomers) must be further stabilized since it exhibits intramolecular instability that causes the destabilization and breakdown of the molecule at both high as well as ambient temperatures. In addition, the molecule, S-adenosyl-1-methionine consists of diastereomers as discussed above. The molecule is diasteromerically unstable both in solution as well as on the shelf. S-adenosyl-1-methionine has therefore been the subject of many patents directed both towards obtaining new stable salts, and towards the provision of preparation processes that can be implemented on an industrial scale.

As used herein, the term “conditions” includes diseases, injuries, disorders, indications and/or afflictions that are associated with decreased levels of S-adenosyl-1-methionine. The term “treat” or “treatment” means that the symptoms associated with one or more conditions associated with low levels of S-adenosyl-1-methionine are alleviated or reduced in severity or frequency and the term “prevent” means that subsequent occurrences of such symptoms are avoided or that the frequency between such occurrences is prolonged.

Typical oral dosages for the treatment of the conditions listed above lie in the range of from 100 mg to 1600 mg or greater per day given in divided doses orally. It is well known in the literature how one may arrive at the optimum dose of S-adenosyl-1-methionine to treat or prevent a particular condition. It is well within the art to determine such doses that in any event will vary from patient population as well as clinical condition to be treated. The dose range discussed above is typical as noted in the literature.

The amount of tryptophan, fumaric acid, azelaic acid, chitosan, dextran or carboxy methyl cellulose that may be used to stabilize the S-adenosyl-1-methionine, its diastereomers or non-racemic mixtures thereof, in the synthetic process of this patent can range from 1% of the weight of the S-adenosyl-1-methionine to be stabilized to 100% of the weight of the S-adenosyl-1-methionine to be stabilized. For reasons of economy, the least amount of chitosan, dextran or carboxy methyl cellulose needed to stabilize the S-adenosyl-1-methionine, its diastereomers or non-racemic mixtures thereof is the preferred quantity. The solutions that result during the synthetic process can be dried by any other methods than freeze drying and that are all well known in the art. After drying, a stable S-adenosyl-1-methionine powder results.

Owing to their simple conception and low costs, the procedures described in this invention easily lend themselves to working out methods of preparation on an industrial scale.

The following examples illustrate the synthetic process by which the new stabilized compositions of S-adenosyl-1-methionine with tryphophan, fumaric acid, azelaic acid, chitosan, dextran or carboxy methyl cellulose may be made. The S-adenosyl-1-methionine used in the following examples may be obtained by any method known in the art, but the preferred method is the one that yields the highest concentration of (S,S) S-adenosyl-1-methionine to (R,S) S-adenosyl-1-methionine irrespective of the methodology. Thus, the preferred method would be one in which the temperature of the extraction, purification and salification processes would be controlled between 2 and 7 degrees C. and the resulting solution would be lyophilized. These examples are given to illustrate the present invention, but not by way of limitation. Accordingly, the scope of this invention should be determined not by the embodiments illustrated, but rather by the appended claims and their legal equivalents.

### EXAMPLE 1

Dissolve 0.5 grams of chitosan in 5 ml of water and add 0.5 grams of S-adenosyl-1-methionine. Stir solution well until completely dissolved and freeze dry.

The sample is left at room temperature in the light for 3 months in a closed bottle with no special protection.

Stability of the new S-adenosyl-1-methionine salts is assessed according to the following protocol:

Isocratic high performance liquid chromatographic analysis of S-adenosylmethionine and S-adenosylhomocysteine in animal tissues: the effect of exposure to nitrous oxide. Bottiglieri, T. (1990) Biomed Chromatogr, 4(6):239-41. An example of the methodology to determine the percentage of diastereomers of S-adenosyl-1-methionine is also well known and a new NMR technique has recently been published. Hanna, Pharmazie, 59, 2004, number 4 pp 251-256.

No S-adenosyl-1-methionine breakdown products is detected, thus showing that the S-adenosyl-1-methionine remained stable for 3 months at room temperature.

### EXAMPLE 2

Dissolve 0.5 grams of chitosan in 5 ml of water and add 0.5 grams of (S,S)-S-adenosyl-1-methionine. Stir solution well until completely dissolved and freeze dry. The sample is left at room temperature in the light for 3 months in a closed bottle with no special protection.

### EXAMPLE 3

Dissolve 0.5 grams of chitosan in 5 ml of water and add 0.5 grams of (R,S)-S-adenosyl-1-methionine. Stir solution well until completely dissolved and freeze dry. The sample is left at room temperature in the light for 12 months in a closed bottle with no special protection.

### EXAMPLE 4

Dissolve 0.5 grams of dextran in 5 ml of water and add 0.5 grams of S-adenosyl-1-methionine. Stir solution well until completely dissolved and freeze dry. The sample is left at room temperature in the light for 3 months in a closed bottle with no special protection.

### EXAMPLE 5

Dissolve 0.5 grams of dextran in 5 ml of water and add 0.5 grams of (S,S)-S-adenosyl-1-methionine. Stir solution well until completely dissolved and freeze dry. The sample is left at room temperature in the light for 3 months in a closed bottle with no special protection.

### EXAMPLE 6

Dissolve 0.5 grams of dextran in 5 ml of water and add 0.5 grams of (R,S)-S-adenosyl-1-methionine. Stir solution well until completely dissolved and freeze dry. The sample is left at room temperature in the light for 3 months in a closed bottle with no special protection.

### EXAMPLE 7

Dissolve 0.5 grams of carboxy methyl cellulose in 5 ml of water and add 0.5 grams of S-adenosyl-1-methionine. Stir solution well until completely dissolved and freeze dry. The sample is left at room temperature in the light for 3 months in a closed bottle with no special protection.

### EXAMPLE 8

Dissolve 0.5 grams of carboxy methyl cellulose in 5 ml of water and add 0.5 grams of (S,S) S-adenosyl-1-methionine. Stir solution well until completely dissolved and freeze dry. The sample is left at room temperature in the light for 3 months in a closed bottle with no special protection.

### EXAMPLE 9

Dissolve 0.5 grams of carboxy methyl cellulose in 5 ml of water and add 0.5 grams of (R,S) S-adenosyl-1-methionine. Stir solution well until completely dissolved and freeze dry. The sample is left at room temperature in the light for 3 months in a closed bottle with no special protection.

### EXAMPLE 10

Dissolve 0.5 grams of carboxy methyl cellulose in 5 ml of water and add 0.5 grams of S-adenosyl-1-methionine tosylate. Stir solution well until completely dissolved and freeze dry. The sample was left at room temperature in the light for 12 months in a closed bottle with no special protection.

HPLC results

| Compound | Solution | |
|---|---|---|
| In H₂O | HPLC retention time | Area of HPLC peak |
| 100 μg/ml | 15.01 min | 1459 |

There were no S-adenosyl-1-methionine breakdown products found. In contrast, however, S-adenosyl-1-methionine 1,4 butanedisulfonate under the same conditions as in example 10 and using the same analytical methods showed a 43.9% deterioration after only four months. S-adenosyl-1-methionine tosylate, under the same conditions as in example 10 showed a 92.9% deterioration after 4 months. (See discussion in U.S. Pat. No. 6,635,615 which is incorporated herein in its entirety.

### EXAMPLE 11

Dissolve 0.5 grams of carboxy methyl cellulose in 5 ml of water and add 0.5 grams of (S,S) S-adenosyl-1-methionine tosylate. Stir solution well until completely dissolved and freeze dry. The sample is left at room temperature in the light for 3 months in a closed bottle with no special protection.

### EXAMPLE 12

Dissolve 0.5 grams of carboxy methyl cellulose in 5 ml of water and add 0.5 grams of (R,S) S-adenosyl-1-methionine tosylate. Stir solution well until completely dissolved and freeze dry. The sample is left at room temperature in the light for 3 months in a closed bottle with no special protection.

### EXAMPLE 13

Dissolve 0.5 grams of carboxy methyl cellulose in 5 ml of water and add 0.5 grams of 1,4 butanedisulfonate S-adenosyl-1-methionine. Stir solution well until completely dissolved and freeze dry. The sample is left at room temperature in the light for 3 months in a closed bottle with no special protection.

### EXAMPLE 14

Dissolve 0.5 grams of carboxy methyl cellulose in 5 ml of water and add 0.5 grams of 1,4 butanedisulfonate (S,S) S-adenosyl-1-methionine. Stir solution well until completely dissolved and freeze dry. The sample is left at room temperature in the light for 3 months in a closed bottle with no special protection.

### EXAMPLE 15

Dissolve 0.5 grams of carboxy methyl cellulose in 5 ml of water and add 0.5 grams of 1,4 butanedisulfonate (R,S) S-adenosyl-1-methionine. Stir solution well until completely dissolved and freeze dry. The sample is left at room temperature in the light for 3 months in a closed bottle with no special protection.

### EXAMPLE 16

Dissolve 0.5 grams of tryptophan in 5 ml of water and add 0.5 grams of S-adenosyl-1-methionine. Stir solution well until completely dissolved and freeze dry. The sample is left at room temperature in the light for 3 months in a closed bottle with no special protection.

### EXAMPLE 17

Dissolve 0.5 grams of tryptophan in 5 ml of water and add 0.5 grams of (S,S) S-adenosyl-1-methionine. Stir solution well until completely dissolved and freeze dry. The sample is left at room temperature in the light for 3 months in a closed bottle with no special protection.

### EXAMPLE 18

Dissolve 0.5 grams of tryptophan in 5 ml of water and add 0.5 grams of (R,S) S-adenosyl-1-methionine. Stir solution well until completely dissolved and freeze dry. The sample is left at room temperature in the light for 3 months in a closed bottle with no special protection.

### EXAMPLE 19

Dissolve 0.5 grams of tryptophan in 5 ml of water and add 0.5 grams of S-adenosyl-1-methionine tosylate. Stir solution well until completely dissolved and freeze dry. The sample is left at room temperature in the light for 3 months in a closed bottle with no special protection.

### EXAMPLE 20

Dissolve 0.5 grams of tryptophan in 5 ml of water and add 0.5 grams of (S,S) S-adenosyl-1-methionine tosylate. Stir solution well until completely dissolved and freeze dry. The sample is left at room temperature in the light for 3 months in a closed bottle with no special protection.

### EXAMPLE 21

Dissolve 0.5 grams of tryptophan in 5 ml of water and add 0.5 grams of (R,S) S-adenosyl-1-methionine tosylate. Stir solution well until completely dissolved and freeze dry. The sample is left at room temperature in the light for 3 months in a closed bottle with no special protection.

### EXAMPLE 22

Dissolve 0.5 grams of tryptophan in 5 ml of water and add 0.5 grams of S-adenosyl-1-methionine 1,4 butanedisulfonate. Stir solution well until completely dissolved and freeze dry. The sample is left at room temperature in the light for 3 months in a closed bottle with no special protection.

### EXAMPLE 23

Dissolve 0.5 grams of tryptophan in 5 ml of water and add 0.5 grams of (S,S) S-adenosyl-1-methionine 1,4 butanedisulfonate. Stir solution well until completely dissolved and freeze dry. The sample is left at room temperature in the light for 3 months in a closed bottle with no special protection.

### EXAMPLE 24

Dissolve 0.5 grams of tryptophan in 5 ml of water and add 0.5 grams of (R,S) S-adenosyl-1-methionine 1,4 butanedisulfonate. Stir solution well until completely dissolved and freeze dry. The sample is left at room temperature in the light for 3 months in a closed bottle with no special protection.

### EXAMPLE 25

Dissolve 0.5 grams of fumaric acid in 5 ml of water and add 0.5 grams of S-adenosyl-1-methionine. Stir solution well until completely dissolved and freeze dry. The sample is left at room temperature in the light for 3 months in a closed bottle with no special protection.

### EXAMPLE 26

Dissolve 0.5 grams of fumaric acid in 5 ml of water and add 0.5 grams of (S,S) S-adenosyl-1-methionine. Stir solution well until completely dissolved and freeze dry. The sample is left at room temperature in the light for 3 months in a closed bottle with no special protection.

### EXAMPLE 27

Dissolve 0.5 grams of fumaric acid in 5 ml of water and add 0.5 grams of (R,S) S-adenosyl-1-methionine. Stir solution well until completely dissolved and freeze dry. The sample is left at room temperature in the light for 3 months in a closed bottle with no special protection.

### EXAMPLE 28

Dissolve 0.5 grams of fumaric acid in 5 ml of water and add 0.5 grams of S-adenosyl-1-methionine tosylate. Stir solution well until completely dissolved and freeze dry. The sample is left at room temperature in the light for 3 months in a closed bottle with no special protection.

### EXAMPLE 29

Dissolve 0.5 grams of fumaric acid in 5 ml of water and add 0.5 grams of (S,S) S-adenosyl-1-methionine tosylate. Stir solution well until completely dissolved and freeze dry. The sample is left at room temperature in the light for 3 months in a closed bottle with no special protection.

### EXAMPLE 30

Dissolve 0.5 grams of fumaric acid in 5 ml of water and add 0.5 grams of (R,S) S-adenosyl-1-methionine tosylate. Stir solution well until completely dissolved and freeze dry. The sample is left at room temperature in the light for 3 months in a closed bottle with no special protection.

### EXAMPLE 31

Dissolve 0.5 grams of fumaric acid in 5 ml of water and add 0.5 grams of S-adenosyl-1-methionine 1,4 butanedisulfonate. Stir solution well until completely dissolved and freeze dry. The sample is left at room temperature in the light for 3 months in a closed bottle with no special protection.

### EXAMPLE 32

Dissolve 0.5 grams of fumaric acid in 5 ml of water and add 0.5 grams of (S,S) S-adenosyl-1-methionine 1,4 butanedisulfonate. Stir solution well until completely dissolved and freeze dry. The sample is left at room temperature in the light for 3 months in a closed bottle with no special protection.

### EXAMPLE 33

Dissolve 0.5 grams of fumaric acid in 5 ml of water and add 0.5 grams of. (R,S) S-adenosyl-1-methionine 1,4 butanedisulfonate. Stir solution well until completely dissolved and freeze dry. The sample is left at room temperature in the light for 3 months in a closed bottle with no special protection.

### EXAMPLE 34

S-adenosyl-1-methionine purification and extraction procedures from yeast are carried out at a temperature of between 2-7.degree. C. These procedures for the extraction and purification are well known in the industry and have been disclosed in the prior art section and are incorporated herein in their entirety by reference. See U.S. Pat. No. 3,893,999, Fiecchi et al for discussion on extraction and purification. Any technique may be used to break the yeast cells to liberate the S-adenosyl-1-methionine but the preferred method is that which is carried out at temperatures between 2 and 7 degrees Centigrade. Yeast cell breakage may be carried out by mechanical means.

Salification of S-adenosyl-1-methionine using 1,4 butanedisulfonic is carried out according to Gennari U.S. Pat. No. 4,465,672 with the exception that the temperature of the procedures is within 2 degrees C. and 7 degrees C. Any pharmaceutically acceptable salt known in the literature to stabilize the molecule may be used. For example, the procedures of Fiecchi or of Gennari (4465672) carried out at the temperatures disclosed in the present patent will result in a (S,S) S-adenosyl-1-methionine/(R,S) S-adenosyl-1-methionine concentration of between 90%-100% (S,S) S-adenosyl-1-methionine vs 10%-0% (R,S) S-adenosyl-1-methionine. When drying of the resulting solution is accomplished by lyophilization, the aformentioned concentration of S-adenosyl-1-methionine diastereomers will remain within the stated range for 5 months. See Hanna for procedures to determine diastereomeric concentrations using NMR. The steps for extraction and purification are outlined below:

They are prepared by a process comprising the following essential stages, which are all critical for the purpose of obtaining a product of absolutely constant and reproducible pharmaceutical purity:
(a) preparing a concentrated aqueous solution of a crude SAM salt by any known method;
(b) purifying the solution by chromatography, by passage through a weakly acid ion exchange resin column;
(c) eluting the SAM with a dilute aqueous solution of the required acid;
(d) titrating the eluate and adjusting the acid quantity to the strictly stoichiometric proportion relative to the SAM present;
(e) concentrating the eluate;
(f) lyophilization.

The aqueous solution prepared in stage (a) can obviously contain any soluble SAM salt because the anion is eliminated in the next passage through the column, and therefore does not interfere with the rest of the process.

In all cases, the pH of the solution is adjusted to between 6 and 7, and preferably. 6.5.

The chromatographic purification stage (b) is carried out preferably with Amberlite IRC50 or Amberlite CG50.

The elution of stage (c) is preferably carried out with a 0.1 N aqueous solution of the required acid.

If titration of the eluate (stage d) shows that the quantity of acid equivalents present is less than 5, this being the usual case, then that quantity of acid corresponding exactly to the deficiency is added in the form of a concentrated commercial aqueous solution. However, if it is shown that an excess of acid is present, this is eliminated by treating the solution with strong basic ion exchange resin in OH.sup.-form, for example Amberlite IRA-401.

In stage (e), the eluate is concentrated to an optimum value for the subsequent lyophilization process, i.e. to a value of between 50 and 100 g/l, and preferably around 70 g/l.

The final lyophilization is carried out by the usual methods, to give a perfectly crystalline salt of 100% purity.

If lyophilization is carried out in the presence of a suitable inert substance, a product is obtained having a smaller residual moisture content, and thus more stable.

More specifically, it has been found that if the prepared salt is intended for use in injectable pharmaceutical forms, lyophilization may be carried out in the presence of, for example, dextran. If however the new salt is intended for the preparation of oral tablets, lyophilization may be carried out in the presence of carboxy methyl cellulose.

### EXAMPLE 35

### See Also Above Example 34

S-adenosyl-1-methionine purification and extraction procedures from bacterial fermentation are also well known see above. However, in order to stabilize the molecule to epimerization, the lower temperatures as disclosed in this patent application are preferable. See U.S. Pat. No. 3,893,999, Fiecchi et al for discussion on purification.

Salification of S-adenosyl-1-methionine using 1,4 butanedisulfonic acid is carried out according to the procedures of Fiecchi or of Gennari U.S. Pat. No. 4,465,672 with the exception that the temperature of the procedures is within 2 degrees C. and 7 degrees C. Any pharmaceutically acceptable salt known in the literature to stabilize the molecule may be used.

For example, the procedures of Gennari carried out at the temperatures disclosed in the present patent will result in a (S,S) S-adenosyl-1-methionine/(R,S) S-adenosyl-1-methionine concentration of between 90%-100% (S,S) S-adenosyl-1-methionine vs 10%-0% (R,S) S-adenosyl-1-methionine. When drying of the resulting solution is accomplished by lyophilization, the aformentioned concentration of S-adenosyl-1-methionine diastereomers will remain within the stated range for 5 months.

See Hanna for procedures to determine diastereomeric concentrations using NMR.

### EXAMPLE 36

Dissolve 0.5 grams of azelaic acid in 5 ml of water and add 0.5 grams of S-adenosyl-1-methionine. Stir solution well until completely dissolved and freeze dry. The sample is left at room temperature in the light for 3 months in a closed bottle with no special protection.

### EXAMPLE 37

Dissolve 0.5 grams of azelaic acid in 5 ml of water and add 0.5 grams of (S,S) S-adenosyl-1-methionine. Stir solution well until completely dissolved and freeze dry. The sample is left at room temperature in the light for 3 months in a closed bottle with no special protection.

### EXAMPLE 38

Dissolve 0.5 grams of azelaic acid in 5 ml of water and add 0.5 grams of (R,S) S-adenosyl-1-methionine. Stir solution well until completely dissolved and freeze dry. The sample is left at room temperature in the light for 3 months in a closed bottle with no special protection.

### EXAMPLE 39

Dissolve 0.5 grams of azelaic acid in 5 ml of water and add 0.5 grams of S-adenosyl-1-methionine tosylate. Stir solution well until completely dissolved and freeze dry. The sample is left at room temperature in the light for 3 months in a closed bottle with no special protection.

### EXAMPLE 40

Dissolve 0.5 grams of azelaic acid in 5 ml of water and add 0.5 grams of (S,S) S-adenosyl-1-methionine tosylate. Stir solution well until completely dissolved and freeze dry. The sample is left at room temperature in the light for 3 months in a closed bottle with no special protection.

### EXAMPLE 41

Dissolve 0.5 grams of azelaic acid in 5 ml of water and add 0.5 grams of (R,S) S-adenosyl-1-methionine tosylate. Stir solution well until completely dissolved and freeze dry. The sample is left at room temperature in the light for 3 months in a closed bottle with no special protection.

### EXAMPLE 42

Dissolve 0.5 grams of azelaic acid in 5 ml of water and add 0.5 grams of S-adenosyl-1-methionine 1,4 butanedisulfonate. Stir solution well until completely dissolved and freeze dry. The sample is left at room temperature in the light for 3 months in a closed bottle with no special protection.

### EXAMPLE 43

Dissolve 0.5 grams of azelaic acid in 5 ml of water and add 0.5 grams of (S,S) S-adenosyl-1-methionine 1,4 butanedisulfonate. Stir solution well until completely dissolved and freeze dry. The sample is left at room temperature in the light for 3 months in a closed bottle with no special protection.

### EXAMPLE 44

Dissolve 0.5 grams of azelaic acid in 5 ml of water and add 0.5 grams of (R,S) S-adenosyl-1-methionine 1,4 butanedisulfonate. Stir solution well until completely dissolved and freeze dry. The sample is left at room temperature in the light for 3 months in a closed bottle with no special protection.

1. A composition comprising S-adenosyl-1-methionine and a member of a group consisting of chitosan, dextran, carboxy methyl cellulose, fumaric acid, azelaic acid, and tryphophan.

2. A composition of claim 1 where the amount of a member of the group consisting of chitosan, dextran, carboxy methyl cellulose, fumaric acid, azelaic acid, and tryphophan is between 0.01% to 100% of the weight of S-adenosyl-1-methionine.

3. A composition comprising a pharmaceutically acceptable salt of S-adenosyl-1-methionine and a member of a group consisting of carboxy methyl cellulose, fumaric acid, azelaic acid, and tryphophan.

4. A composition of claim 3 wherein the S-adenosylmethionine salt is selected from the group consisting of S-adenosyl-1-methionine tosylate bisulfate, S-adenosyl-1-methionine-1,4 butanedisulfonate, S-adenosyl-1-methionine sulfate, S-adenosyl-1-methionine tosylate.

5. A composition of claim 3 wherein the amount of carboxy methyl cellulose, fumaric acid, tryphophan and azelaic acid is between 0.01% to 100% of the weight of the S-adenosyl-1-methionine salt.

6. A composition of claims 1, 3 and 4 wherein S-adenosyl-1-methionine is selected from the group consisting of the optically pure diastereomer (S,S) S-adenosyl-1-methionine or a defined non-racemic ratio of (S,S) S-adenosyl-1-methionine and (R,S) S-adenosyl-1-methionine.

7. A composition of claim 6 wherein the defined non-racemic ratio of (S,S) S-adenosyl-1-methionine: (R,S) S-adenosyl-1-methionine is about 1%:100% to about 100%:1% by weight.

8. A composition useful for the treatment of depression, osteoarthritis, or of conditions in which lowered levels of methylation in genomic DNA or RNA, cell, tissue or blood play a role in pathology, comprising an effective amount of S-adenosyl-1-methionine produced by yeast fermentation, extracted and purified by methods known in the art in the temperature range of between 2 and 7 degrees centigrade to maintain defined non-racemic ratio of (S,S) S-adenosyl-1-methionine to (R,S) S-adenosyl-1-methionine between 100%−70%/0%−30% respectively, and salified using a pharmaceutically acceptable acid to stabilize the resulting defined non-racemic ratio of (S,S) S-adenosyl-1-methionine to (R,S) S-adenosyl-1-methionine and then drying the resulting solution to obtain a stable powder.

9. A composition of claim 8 wherein the pharmaceutically acceptable acid used to stabilize the S-adenosyl-1-methionine is 1,4-butanedisulphonic acid.

10. A composition useful for the treatment of depression, osteoarthritis, or of conditions in which lowered levels of methylation in genomic DNA or RNA, cell, tissue or blood play a role in pathology, comprising an effective amount of S-adenosyl-1-methionine produced by yeast fermentation, extracted and purified by methods known in the art in the temperature range of between 2 and 7 degrees centigrade to maintain defined non-racemic-ratio of (S,S) S-adenosyl-1-methionine to (R,S) S-adenosyl-1-methionine between 96.9%-80%/3.1%-20% respectively, and salified using a pharmaceutically acceptable acid to stabilize the resulting defined non-racemic ratio of (S,S) S-adenosyl-1-methionine to (R,S) S-adenosyl-1-methionine and then drying the resulting solution to obtain a stable powder.

11. A composition of claim 10 wherein the pharmaceutically acceptable acid used to stabilize the S-adenosyl-1-methionine is selected from the group consisting of sulphuric acid and paratoluensulphonic acid.

12. A composition useful for the treatment of depression, osteoarthritis, or of conditions in which lowered levels of methylation in genomic DNA or RNA, cell, tissue or blood play a role in pathology, comprising an effective amount of S-adenosyl-1-methionine produced by bacterial fermentation, extracted and purified in the temperature range of between 2 and 7 degrees centigrade to maintain defined non-racemic ratio of (S,S) S-adenosyl-1-methionine to (R,S) S-adenosyl-1-methionine between 100%-70%/0%-30% respectively, and salified using a pharmaceutically acceptable acid to stabilize the resulting defined non-racemic ratio of (S,S) S-adenosyl-1-methionine to (R,S) S-adenosyl-1-methionine and then drying the resulting solution to obtain a stable powder.

13. A composition of claim 12 wherein the pharmaceutically acceptable acid used to stabilize the S-adenosyl-1-methionine is selected from the group consisting of 1,4-butanedisulphonic acid, sulphuric acid and paratoluensulphonic acid.
